(19) United States
(12) Patent Application Publication
(10) Pub. No.: US 2017/0209113 A1
(43) Pub. Date: Jul. 27, 2017

(54) METHODS AND SYSTEMS FOR ADAPTIVE SCAN CONTROL

(71) Applicant: General Electric Company, Schenectady, NY (US)

(72) Inventors: John Irvin Jackson, Brookfield, WI (US); Darin Robert Okerlund, Muskego, WI (US); Christine Carol Hammond, Oconomowoc, WI (US)

(21) Appl. No.: 15/004,155

(22) Filed: Jan. 22, 2016

(51) Int. Cl. A61B 6/00; A61B 5/0456; A61B 6/04; A61B 6/03; A61B 5/024

(52) U.S. Cl. CPC A61B 6/541; A61B 6/032; A61B 6/504; A61B 5/024; A61B 6/5211; A61B 6/0407; A61B 6/4266; A61B 6/4435; A61B 5/0456

(57) ABSTRACT

Methods and systems are provided for adaptive scan control. In one embodiment, a method comprises, during a scan session, performing a first scan of a heart of a subject using a first scan protocol, performing a second scan of the heart using a second scan protocol, and performing a third scan of the heart using the first scan protocol, and while performing the first scan and the third scan, adjusting a scan rate of the first scan protocol based on a heart rate of the subject. In this way, multiple scan protocols, such as angiography and perfusion scan protocols, can be interleaved within a single scan and the scan protocol may be adapted to a patient.

### FIELD

Embodiments of the subject matter disclosed herein relate to non-invasive diagnostic imaging, and more particularly, to real-time adaptive scanning.

### BACKGROUND

Non-invasive imaging technologies allow images of the internal structures of a patient or object to be obtained without performing an invasive procedure on the patient or object. In particular, technologies such as computed tomography (CT) use various physical principals, such as the differential transmission of x-rays through the target volume, to acquire image data and to construct tomographic images (e.g., three-dimensional representations of the interior of the human body or of other imaged structures).

Cardiac CT angiography (CTA) scans are designed for visualization of the coronary arteries, with areas of narrowing (stenoses) and any associated plaque, as well as the presence and amount of calcium. Cardiac CT perfusion (CTP) scans are designed for visualization of the contrast agent in the myocardium, especially to identify areas which are poorly perfused (and hence have a delayed and/or reduced contrast uptake) relative to areas of normal perfusion.

Typically cardiac CTA and CTP scans are performed with independent scan sequences, and with separate contrast agent injections. As such, this may result in a longer time for such exams, as there may be considerable wait times of several minutes between CTA and CTP, for example.

### BRIEF DESCRIPTION

Methods and systems are provided for adaptive scan control. In one embodiment, a method comprises, during a scan session, performing a first scan of a heart of a subject using a first scan protocol, performing a second scan of the heart using a second scan protocol, performing a third scan of the heart using the first scan protocol, and while performing the first scan and the third scan, adjusting a scan rate of the first scan protocol based on a heart rate of the subject. In this way, multiple scan protocols, such as angiography and perfusion scan protocols, can be interleaved within a single scan and radiation dose delivered to the patient may be reduced. Furthermore, by adaptively changing the CTP and CTA protocols based on the heart rate, any variations in the scans due to changes in heart rate (during an arrhythmia, for example) may be reduced.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a pictorial view of an imaging system according to an embodiment of the invention.

FIG. 2 shows a block schematic diagram of an exemplary imaging system according to an embodiment of the invention.

FIG. 3 shows a high-level flow chart illustrating an example method for adaptively combining cardiac CT perfusion (CTP) and CT angiography (CTA) scans based on a heart rate of a subject into a single scan protocol according to an embodiment of the invention.

FIG. 4 shows a table including example scan parameters for the scan protocol.

FIG. 5 shows a high-level flow chart illustrating an example method for adjusting the scan protocol based on a measured contrast level and the heart rate.

FIG. 6 shows a set of graphs illustrating an example control of an imaging system according to an embodiment of the invention.

### DETAILED DESCRIPTION

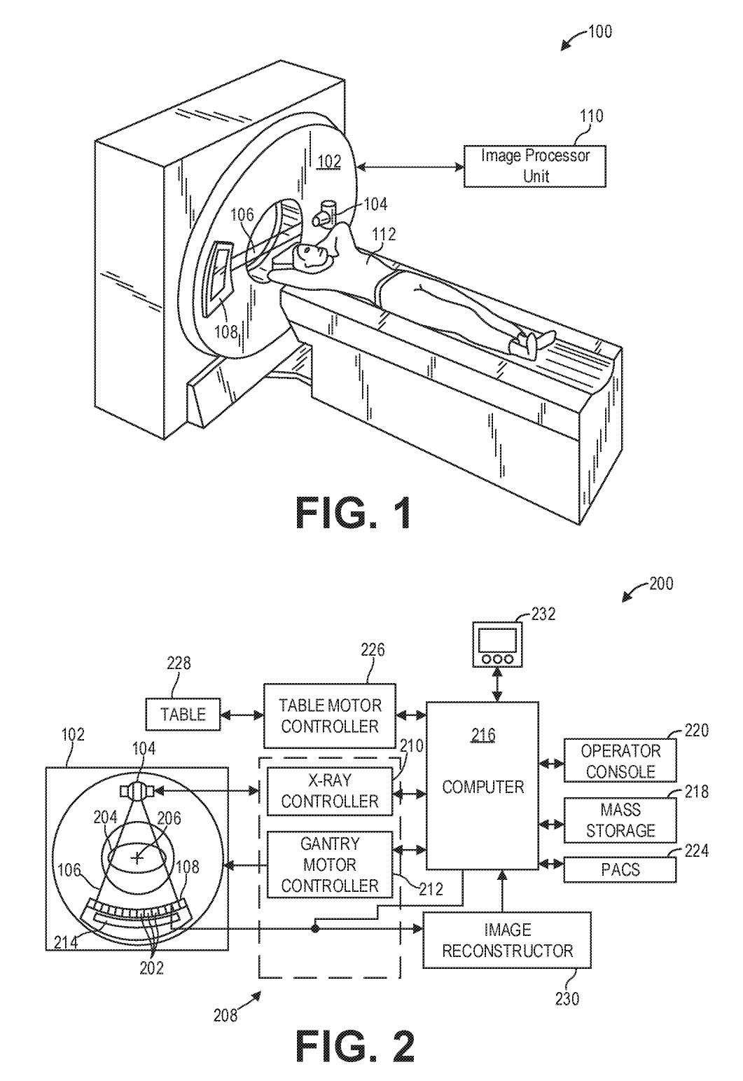

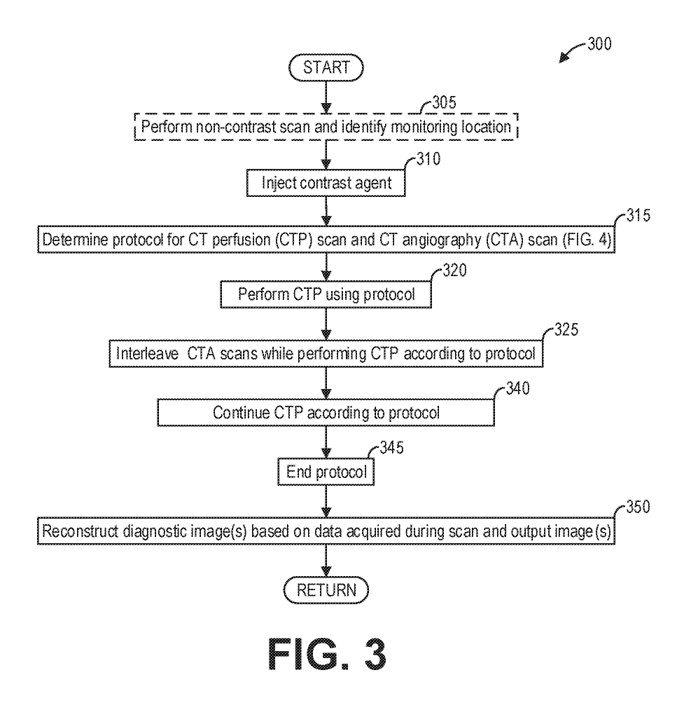

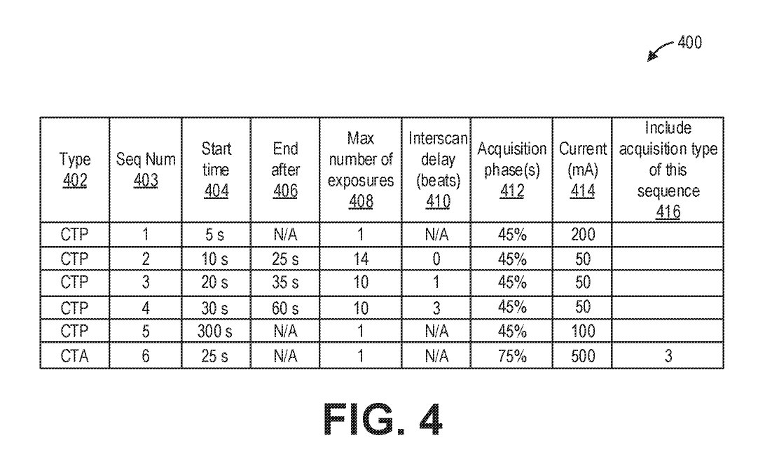

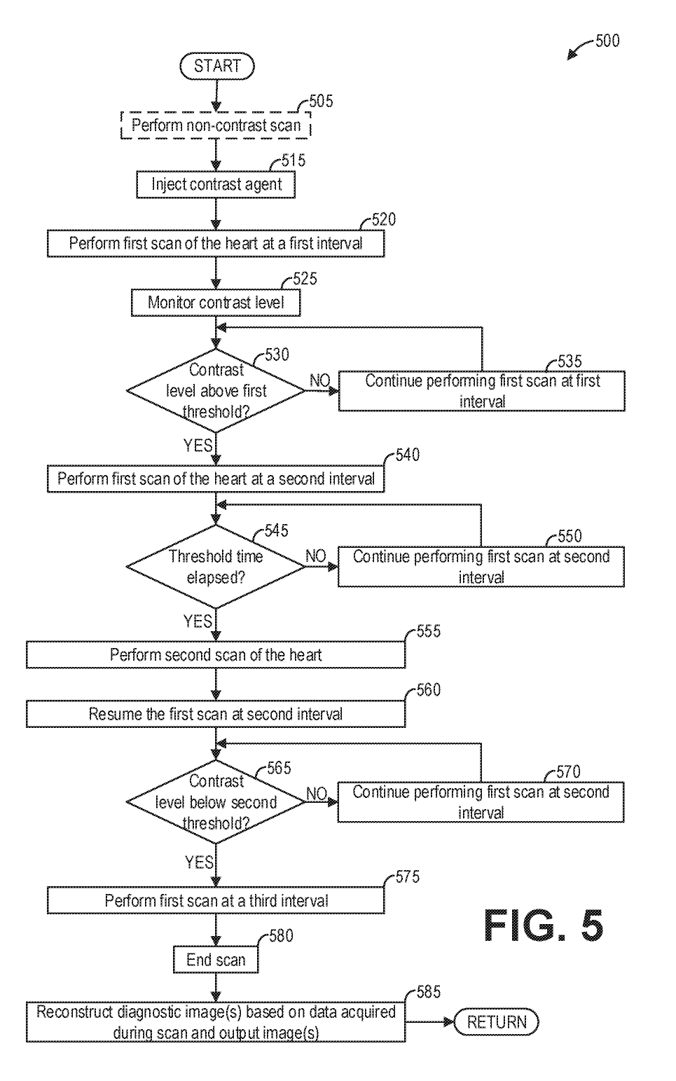

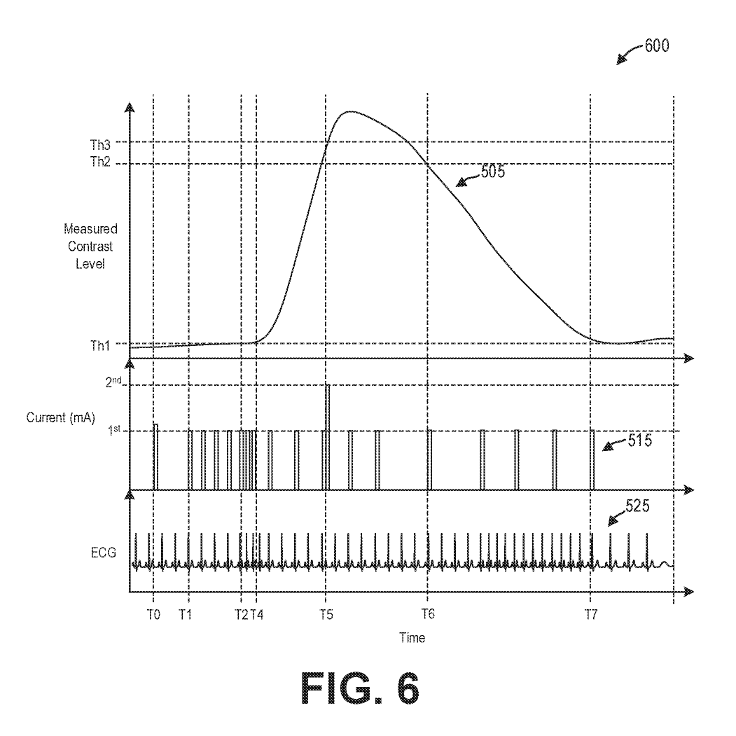

The following description relates to various embodiments of medical imaging systems. In particular, methods and systems are provided for adaptively controlling a diagnostic scan by monitoring contrast enhancement. An example of a computed tomography (CT) imaging system that may be used to acquire images processed in accordance with the present techniques is provided in FIGS. 1 and 2. A method for adaptive scan control, such as the method shown in FIG. 3, may include monitoring one or more of contrast levels and heart rate of a subject during a scan and adjusting scan parameters responsive thereto. Such a method enables personalization of scan protocols on a patient-by-patient basis. Furthermore, by adapting scans based on monitored contrast levels and heart rates in real-time, multiple scan protocols may be combined into a single scan. As an example, a method, such as the method depicted in FIG. 5, includes interleaving CT angiography (CTA) and CT perfusion (CTP) scans into a single scan by switching scan protocols responsive to contrast levels measured during the scan. An operator of the CT imaging system may manually intervene in the automatic adjustment of scan parameters and adjust the scan parameters; examples of the scan parameters are shown in a table in FIG. 4. Transitions between different stages of a multi-protocol scan may be triggered based on levels and slopes of multiple contrast curves and electrocardiogram (ECG), as depicted in FIG. 6.

Though a CT system is described by way of example, it should be understood that the present techniques may also be useful when applied to images acquired using other imaging modalities, such as tomosynthesis, MM, C-arm angiography, and so forth. The present discussion of a CT imaging modality is provided merely as an example of one suitable imaging modality.

As used herein, the phrase “pixel” also includes embodiments of the invention where the data is represented by a “voxel.” Thus, both the terms “pixel” and “voxel” may be used interchangeably herein.

Also as used herein, the phrase “reconstructing an image” is not intended to exclude embodiments of the present invention in which data representing an image is generated, but a viewable image is not. Therefore, as used herein, the term “image” broadly refers to both viewable images and data representing a viewable image. However, many embodiments generate (or are configured to generate) at least one viewable image.

Various embodiments may be implemented in connection with different types of imaging systems. For example, various embodiments may be implemented in connection with a CT imaging system in which an x-ray source projects a fan- or cone-shaped beam that is collimated to lie within an x-y plane of a Cartesian coordinate system and generally referred to as an “imaging plane.” The x-ray beam passes through an object being imaged, such as a patient. The beam, after being attenuated by the object, impinges upon an array of radiation detectors. The intensity of the attenuated radiation beam received at the detector array is dependent upon the attenuation of an x-ray beam by the object. Each detector element of the array produces a separate electrical signal that is a measurement of the beam intensity at the detector location. The intensity measurement from all the detectors is acquired separately to produce a transmission profile.

In third-generation CT systems, the x-ray source and the detector array are rotated with a gantry within the imaging plane and around the object to be imaged such that the angle at which the x-ray beam intersects the object constantly changes. A complete gantry rotation occurs when the gantry concludes one full 360 degree revolution. A group of x-ray attenuation measurements (e.g., projection data) from the detector array at one gantry angle is referred to as a “view.” A view is, therefore, each incremental position of the gantry. A “scan” of the object comprises a set of views made at different gantry angles, or view angles, during one revolution of the x-ray source and detector. Further, “short scan” images may also be reconstructed from a set of views acquired over less than a full gantry rotation.

In an axial scan, the projection data is processed to construct an image that corresponds to a two-dimensional slice taken through the object. One method for reconstructing an image from a set of projection data is referred to in the art as a filtered backprojection technique. This process converts the attenuation measurements from a scan into integers called “CT numbers” or “Hounsfield units” (HU), which are used to control the brightness of a corresponding pixel on, for example, a liquid-crystal display (LCD) flat panel monitor.

FIG. 1 illustrates an exemplary CT system 100 configured to allow fast and iterative image reconstruction. Particularly, the CT system 100 is configured to image a subject such as a patient, an inanimate object, one or more manufactured parts, and/or foreign objects such as dental implants, stents, and/or contrast agents present within the body. In one embodiment, the CT system 100 includes a gantry 102, which in turn, may further include at least one x-ray radiation source 104 configured to project a beam of x-ray radiation 106 for use in imaging the patient. Specifically, the radiation source 104 is configured to project the x-rays 106 towards a detector array 108 positioned on the opposite side of the gantry 102. Although FIG. 1 depicts only a single radiation source 104, in certain embodiments, multiple radiation sources may be employed to project a plurality of x-rays 106 for acquiring projection data corresponding to the patient at different energy levels to increase the scanned volume size, or to scan a volume more quickly.

In certain embodiments, the CT system 100 further includes an image processing unit 110 configured to reconstruct images of a target volume of the patient using an iterative or analytic image reconstruction method. For example, the image processing unit 110 may use an analytic image reconstruction approach such as filtered backprojection (FBP) to reconstruct images of a target volume of the patient. As another example, the image processing unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the patient.

FIG. 2 illustrates an exemplary imaging system 200 similar to the CT system 100 of FIG. 1. In accordance with aspects of the present disclosure, the system 200 is configured to reconstruct images with a user-specified temporal window in real-time. In one embodiment, the system 200 includes the detector array 108 (see FIG. 1). The detector array 108 further includes a plurality of detector elements 202 that together sense the x-ray beams 106 (see FIG. 1) that pass through a subject 204 such as a patient to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along an arc of a circle.

In one embodiment, the system 200 includes a control mechanism 208 to control movement of the components such as rotation of the gantry 102 and the operation of the x-ray radiation source 104. In certain embodiments, the control mechanism 208 further includes an x-ray controller 210 configured to provide power and timing signals to the radiation source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The data sampled and digitized by the DAS 214 is transmitted to a computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage device.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates only one operator console 220, more than one operator console may be coupled to the system 200, for example, for inputting or outputting system parameters, requesting examinations, and/or viewing images. Further, in certain embodiments, the system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks.

In one embodiment, for example, the system 200 either includes, or is coupled to a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a motorized table 228. Particularly, the table motor controller 226 moves the table 228 to appropriately position the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the system 200 and instead the computing device 216 may perform one or more functions of the image reconstructor 230. Moreover, the image reconstructor 230 may be located locally or remotely, and may be operatively connected to the system 100 using a wired or wireless network. Particularly, one exemplary embodiment may use computing resources in a “cloud” network cluster for the image reconstructor 230.

In one embodiment, the image reconstructor 230 reconstructs the images stored in the storage device 218. Alternatively, the image reconstructor 230 transmits the reconstructed images to the computing device 216 for generating useful patient information for diagnosis and evaluation. In certain embodiments, the computing device 216 transmits the reconstructed images and/or the patient information to a display 232 communicatively coupled to the computing device 216 and/or the image reconstructor 230.

The various methods and processes described further herein may be stored as executable instructions in non-transitory memory on a computing device in system 200. In one embodiment, image reconstructor 230 may include such instructions in non-transitory memory, and may apply the methods described herein to reconstruct an image from scanning data. In another embodiment, computing device 216 may include the instructions in non-transitory memory, and may apply the methods described herein, at least in part, to a reconstructed image after receiving the reconstructed image from image reconstructor 230. In yet another embodiment, the methods and processes described herein may be distributed across image reconstructor 230 and computing device 216.

In one embodiment, the display 232 allows the operator to evaluate the imaged anatomy. The display 232 may also allow the operator to select a volume of interest (VOI) and/or request patient information, for example, via graphical user interface (GUI) for a subsequent scan or processing.

Typically, cardiac CT angiography (CTA) scans are designed for visualization of the coronary arteries and CT perfusion (CTP) scans are designed for visualization of the contrast agent in the myocardium. Typically, analysis may be quantitative, trying to identify localized myocardial blood flow rates and/or volumes, or qualitative. There may be a series of CTP exposures (dynamic scanning) or a very limited number of exposures (typically one) looking for areas with brightness differences. In the latter case, timing may be attempted that may highlight differences between healthy and ischemic myocardium.

Currently cardiac CT angiography (CTA) and CT perfusion (CTP) scans are performed with independent scan sequences, with separate contrast agent injections. This may result in a longer time for such exams (need to wait several minutes between CTA and CTP), increased contrast agent use (cost and patient renal impact), and slightly increased patient radiation dose because the CTA scan may not be able to be incorporated into the CTP analysis. As such, cardiac scans have some unique challenges that may need to account for changes in the patient's heart rate (either increasing, decreasing, or having some arrhythmias), and the heart rate changes may lead to sub-optimal scan timing, for example.

FIG. 3 shows a high-level flow chart illustrating an example method 300 for adaptively combining cardiac CT perfusion (CTP) and CT angiography (CTA) scans based on a heart rate of a subject into a single scan protocol. Method 300 may be carried out by the components and systems depicted in FIGS. 1 and 2, however it should be understood that the method may be implemented on other components and systems not depicted without departing from the scope of the present disclosure.

Method 300 may begin at 305. At 305, method 300 may optionally include performing a non-contrast scan and identifying a monitoring location. The non-contrast scan may be taken to establish a baseline image for the area to be monitored before delivery of a contrast agent. The baseline image may then be used to align the patient and the region of interest within the imaging device. For cardiac scans, the monitoring location comprises the heart of the patient wherein contrast level is monitored during the scan. Furthermore, the monitoring location may be positioned within the imaging area such that the projection data acquired for diagnostic purposes may also be used for monitoring. Thus, an operator may select the monitoring location based on the baseline image acquired. Determining the monitoring location may therefore comprise receiving a selection of a monitoring location from an operator, for example via operator console 220.

At 310, method 300 includes injecting a contrast agent into the patient. As a non-limiting example, the contrast agent may comprise iodine. As other examples, the contrast agent may comprise an ionic contrast medium such as meglucamine diatriozoate, or a non-ionic contrast medium such as iopromide or ohexol. The contrast agent may be intravenously injected using either automatic or manual methods.

At 310, method 300 includes determining a protocol for combined CTP and CTA scans. The protocol may include scan parameters. Herein, the scan parameters may include, but are not limited to, slice thickness, reconstruction interval, pitch, table speed, scan delay, and so on. The scan parameters may be predetermined according to various methods. For example, an operator may manually set the scan parameters based on experience. As another example, a prediction model may automatically determine the scan parameters based on, for example, the anatomical part being imaged and patient-specific data. The scan parameters may further be determined based on contrast administration, including but not limited to iodine concentration of the contrast agent, injection flow rate (e.g., amount of contrast delivered per unit time), injection duration (e.g., contrast volume), and so on.

The protocol may further include timing information such as the desired delay from the contrast injection to CTA exposure. As an example, a timing bolus scan may be included to determine the desired delay from the contrast injection to the CTA exposure. Based on the timing bolus response, and a nominal expected heart rate, the protocol may determine a sequence of CTP exposures. As such, the protocol may include parameters such as sequence of CTP and CTA scans, and start time, scan intervals, number of exposures, acquisition phase of each of the CTP and CTA scans, and the like. As such, the parameters may be determined based on a model, or adaptively looked up from a look-up table, or may be entered by a user. An example set of parameters for an example protocol including a combination of CTP and CTA scans is shown in FIG. 4.

Turning now to FIG. 4, table 400 shows parameters for a sample or example protocol. Herein, the example protocol includes a baseline scan, followed by rapid scanning to determine a contrast arrival time, subsequently followed by a slightly slower scanning during increasing contrast and start of decrease, then ending with significantly slower scanning. The parameters are listed in a table form for illustrative purposes only.

Table 400 includes several fields (or columns) 402 through 416, each of which includes the parameters pertaining to the example protocol. Each row of the table includes a sequence of the protocol.

The first row of the table includes CTP scan in field 402, with a sequence number 1 in field 403 indicating that the CTP scan is performed at the beginning of the protocol. As described earlier, CTP scans are designed for visualization of the contrast agent in the myocardium, especially to identify areas which are poorly perfused (and hence have a delayed and/or reduced contrast uptake) relative to areas are normal perfusion.

For the first CTP scan, the start time in field 404 is 5 sec, indicating that the CTP scan will begin at 5 sec. Field 406 includes the end time, which for the first scan is “N/A” since the first CTP scan includes only one exposure (field 408). The first CTP scan may be performed by setting the source current to 200 mA and may be acquired at the acquisition phase of 45% of the R-R interval. As such, the R-R interval is the inter-beat interval of the heart, which is typically determined based on electrocardiogram (ECG) output of the patient. The system may monitor the ECG output and may predict the R-R interval and adaptively adjust the scan time in order to scan at the appropriate acquisition phase, for example.

Thus, the first CTP scan may be a moderately-high quality scan which may be regarded as a baseline scan. Continuing on with the protocol, the next sequence may include a sequence of CTP scans, as indicated in the second row of the table. However, the second CTP scan may start at 10 sec, and end at 25 sec following 14 exposures with a source current of 50 mA. Herein, the inter-scan delay, measured in beats, is zero, indicating that the CTP scan is performed on consecutive heart beats, and further each of the CTP scans is performed during 45% of the R-R interval (field 412). Thus, multiple scans are performed in the second sequence. The rapid scanning may allow the system to determine a contrast arrival time, for example.

However, the second sequence may be interrupted by a third sequence of CTP scans starting at 20 sec, as indicated in the third row of the table. In the table shown in FIG. 4, scan sequences with higher sequence number may have a higher priority over scans with lower sequence number.

Thus, the second CTP scan sequence may be interrupted briefly, and system may perform the third sequence of CTP scans. Herein, the third CTP scan is a sequence of 10 scans starting at 20 sec, and ending at 35 sec, where every other heart beat is scanned. For example, inter-scan delay of 1 indicates that the scan is performed on a first heart-beat, then skips the next consecutive heart-beat, and performs a scan on the third successive heart-beat of the cardiac cycle.

However, the third scan sequence may further be interrupted by a fourth CTP scan sequence starting at 30 sec, and lasting until 60 sec, including a series of 10 exposures with a source current of 50 mA acquired at 45% of R-R interval, wherein the scans are performed every fourth beat. The fourth CTP scan sequence may be followed by a fifth CTP scan performed at 5 min or 300 sec with a source current of 100 mA.

Furthermore, at 25 sec while the system is performing the third sequence, the sequence may be interrupted to perform the sixth sequence, which is a CTA scan. As described earlier, cardiac CTA scans are designed for visualization of the coronary arteries, with areas of narrowing (stenoses) and any associated plaque, as well as the presence and amount of calcium. Herein, the CTA scan begins at 25 sec, and a single scan is performed with a higher source current of 500 mA, and the scan is performed at 75% of the R-R interval. Upon completion of the CTA scan the system would return to complete any and all of the remaining exposures from sequence 3, until the end time is reached (35 sec in this example), or until interrupted by another sequence. Further to the CTA scan at a current of 500 mA exposure surrounding 75%, the CTA scan may further include a second CTA scan at 50 mA exposure at 45%, for example.

The table 400 shows an example scanning protocol that be used to combine CTA and CTP scan in a single scan sequence. In the example shown in table 400, there is a nominal plan, with changes or transitions based on times or beat counts. Alternatively, sequences or rules may be defined and enacted so that the scan prescription may be developed in real time to achieve a similar effect. Herein, a user may be able to actively adjust the sequence based on real-time ECG data of the patient, for example. In some examples, the transition between each scan sequence may be adaptively adjusted based on contrast levels. For example, the set of CTA scans may be interleaved into the CTP scan sequence when the contrast level reaches a threshold contrast.

In some embodiments, a user interface may display the protocol, and the user may adaptively change a large number of acquisition and time settings, as shown in the table above. In some example embodiments, a more limited and streamlined display may be used, with rules or default values being used for settings that are not explicitly defined.

Thus, an example scan protocol is shown in FIG. 4. Herein, the protocol may include a baseline scan, following by rapid scans, followed by a slower scanning during increasing contrast and decreasing contrast, and subsequently followed by a significantly slower scan. Herein, the CTP and CTA scans are triggered by the heart rate. As described earlier, other protocols may be used to combine the CTP and CTA scanning. Returning to FIG. 3, at 315 of method 300, the protocol for CTP and CTA scan sequence may be determined. Next, at 320 of method 300, the CTP scan may be performed according to the protocol determined at 315. For the example protocol shown in table 400 of FIG. 4, the first sequence including a moderately-high quality CTP scan may be started at 5 sec. The rest of the protocol may be performed as described with reference to FIG. 4.

Method 300 proceeds to 325, where CTA scans may be interleaved while performing the CTP scans according to the protocol determined at 315. For the example protocol shown in FIG. 4, the sequence of CTP scans may be interrupted at 25 sec into the scanning, and the system may perform a CTA scan at 75% of the R-R interval at a source current of 500 mA. Upon completing the CTA scan sequence at 325, method 300 proceeds to 340 where the remainder of the CTP scans of the protocol may be continued. As described with reference to the example protocol in table 400 of FIG. 4, the CTA scan may be started at 25 sec, and upon completion, the protocol may continue with the third scan which includes a sequence of 10 CTP scans occurring for every other heartbeat. The protocol may continue on until all the scan sequences of the protocol are completed.

Once the sequences of the protocol are completed, method 300 proceeds to 345, where the protocol may be ended. Proceeding to 350, method 300 includes reconstructing one or more diagnostic images based on data acquired during the scan. The one or more diagnostic images may be reconstructed using known reconstruction techniques, such as filtered back projection or iterative reconstruction. Furthermore, at 350, method 300 includes outputting the one or more diagnostic images. As non-limiting examples, outputting the one or more diagnostic images may comprise outputting the one or more diagnostic images to a display device (e.g., display device 232) for display to an operator or a physician, to a storage medium (e.g., mass storage 218) for retrieving at a later time, and so on. Method 300 may then end.

Thus an example method for a combined cardiac CTA/CTP scan sequence is shown. Some scan parameters may be changed for the CTA scan, but other parameters may be maintained to reduce inter-scan delays. Herein, the initiation for the CTA may be determined based on a timer, and may further depend on completion of a certain number of prior CTP scans. However, initiation for the CTA scan may be determined by a real-time assessment of a contrast agent level, rather than the completion of a fixed number of prior perfusion scans as described below with reference to FIG. 5.

FIG. 5 shows a high-level flow chart illustrating an example method 500 for adjusting the scan protocol based on a measured contrast level and the heart rate according to an embodiment. In particular, method 500 relates to interleaving a perfusion scan and an angiography scan by monitoring a contrast level and adjusting scan parameters based on the monitored contrast level. Method 500 may be carried out by the components and systems depicted in FIGS. 1 and 2, however it should be understood that the method may be implemented on other components and systems not depicted without departing from the scope of the present disclosure.

Method 500 may begin at 405. At 405, method 400 includes performing a non-contrast scan of the target volume or region of interest (e.g., the heart of the patient). Performing the non-contrast scan includes acquisition of projection data as well as the reconstruction of the acquired projection data into one or more images.

Furthermore, by way of such a scan, images are acquired at positions in the scan range where there is no contrast agent. Thus, the non-contrast scan may comprise a baseline scan which establishes baseline contrast values (i.e., contrast levels prior to contrast injection) in a monitoring region.

After performing the non-contrast scan, method 500 proceeds to 515. At 515, method 500 includes injecting a contrast agent. The contrast agent may be manually or automatically intravenously injected into the patient. The contrast agent may be an imaging enhancing agent, a biomedical agent, a blood agent, a nonionic contrast agent, an iodinated contrast agent, and so on.

After injecting the contrast agent, method 500 proceeds to 520. At 520, method 500 includes performing a first scan of the heart at a first interval. As an example, the first scan may be a perfusion scan. Performing a perfusion scan comprises scanning the patient according to perfusion scan parameters, including but not limited to radiation dosage, current settings, acquisition phase, in order to generate one or more perfusion maps and determine various perfusion parameters such as blood flow, blood volume, mean transit time, and so on. The first interval comprises an amount of time between scans, and may be determined based on the timing delay from contrast injection to contrast arrival at the location of interest (heart for example). In addition, the first interval may be adjusted based on heart beat interval, or the R-R interval. For example, the first interval may include an inter scan delay of 0, indicating that perfusion scans may be performed during every consecutive beat.

While method 400 performs the perfusion scan, the method also monitors contrast levels in real-time by processing the acquired projection data. Specifically, at 525, method 500 includes monitoring a contrast level of the heart based on the perfusion scan data. Monitoring the contrast level of the heart based on the perfusion scan data may comprise, as a non-limiting example, reconstructing an image of at least the heart based on the perfusion scan data and evaluating the contrast or HU level of the image. In some examples, method 500 may reconstruct only one or two slices to monitor the contrast levels. However, in other examples, method 500 may reconstruct the full volume to monitor the contrast levels.

At 530, method 500 includes determining if the contrast level is above a first threshold. For example, if a threshold level is detected in the right ventricle or pulmonary artery, then method 300 may determine that the contrast level is above the first threshold and proceed to 540. In some examples the first threshold may comprise a vector indicating a scalar amount of contrast as well as a direction indicating that the increase in contrast is reaching a maximum. Further, in some examples the method automatically determines whether the contrast level has reached the first threshold. Alternatively or additionally, an operator of the imaging apparatus may manually indicate, based on a review of the contrast curves, that the contrast enhancement is reaching a maximum by selecting a button via an operator console and/or a display device.

If the contrast level is below the first threshold (“NO”), then method 500 may proceed to 535 where the first scan may be continued at the first interval and then return to 530. If the contrast level is above the first threshold (“YES”), then method 500 proceeds to 540.

At 540, method 500 includes performing a first scan at a second heart interval. Herein, the first scan may be a perfusion scan, and the second interval may be different from the first interval. Similar to the first interval, the second interval may be based on the heart rate. As an example, scanning may occur every other heart beat at 540.

At 545, method 500 includes checking if threshold time has elapsed. In some examples, it may be determined if a threshold number of scans have completed. In some other examples, it may be determined if a threshold metric is crossed (for example, when the contrast level is at a maximum). If “NO” then the method proceeds to 550 where the first scan may be continued at the second interval, and the method may return to 545.

However, if threshold time has elapsed (or threshold number of scans are completed, or threshold contrast levels are reached), then the method proceeds to 555 where a second scan may be performed on the heart. The second scan may be an angiography scan. To perform the angiography scan, the method adjusts multiple scan parameters, including but not limited to dose, acquisition phase, source current, and so on. The second scan may include a single CTA scan. In some examples, the second scan may include a sequence of CTA scans, wherein the different CTA scans may have different scanning parameters.

Upon completion of the second scan, method 500 proceeds to 560 where the first scan may be resumed. For example, the CTP scan may be resumed at the second interval.

At 565, method 500 includes determining if the contrast level is below a second threshold. The second threshold is established such that when the contrast level reaches the second threshold, the contrast level is exiting peak contrast enhancement. In some examples, if the contrast level decreases from a peak by more than 20 HU, then the method may return a “YES”. In some more examples the second threshold may comprise a vector indicating a scalar amount of contrast as well as a direction indicating that the contrast is decreasing away from peak contrast enhancement. Further, in some examples the method automatically determines whether the contrast level has reached the second threshold. Alternatively or additionally, an operator of the imaging apparatus may manually indicate that the contrast level is decreasing away from the maximum by selecting a button via an operator console and/or a display device.

If the contrast level is above the second threshold (“NO”), method 500 proceeds to 570 where the first scan may be continued at the second interval, and the method returns to 565.

However, if the contrast level is below the second threshold (“YES”), method 500 proceeds to 575. At 575, method 500 includes performing the first scan at a third interval. As before, the first scan may be a perfusion scan. To perform the perfusion scan, the method adjusts one or more scan parameters. Furthermore, the scan parameters may be different than the scan parameters used for the perfusion scan performed at each of 520 and 540. The third interval may be different from each of the first interval and the second interval. As an example, scanning may occur every fourth heartbeat. The scan ends at 580.

At 585, method 500 includes reconstructing and outputting diagnostic images based on the perfusion scan data and the angiography scan data as well as computing perfusion parameters. Method 500 may then end.

The transitions between the CTP and CTA scans may be calculated in real time based on scan data analysis, or transitions may be manually forced by an input from the user.

As described earlier, the transitions between the acquisition phases of the CTP and CTA scans may be controlled in numerous ways that provide flexibility for a wide range of patients. A further challenge with CTP scans is that it is often desirable to have the system scan as rapidly as possible, such as every heartbeat, but, depending on the patient's heart rate, the system may or may not be able to scan as rapidly, but may be able to at best scan every other heartbeat. For example, if an x-ray exposure is 0.25 seconds and the x-ray system requires 0.48 seconds between exposures to complete the data handling associated with that exposure and set up for the subsequent exposure, then the system can scan every beat with a heart rate of 82 bpm (0.732 sec per beat, which is greater than 0.25+0.48), but could only scan every other beat at 83 bpm (0.723 sec per beat, which is less than 0.25+0.48). Thus, in a 10 second interval, there could be 13 or 14 exposures, or 6 or 7 exposures. Furthermore, if the heart rate is slightly varying from beat to beat, the system may scan consecutive beats for some beats, and need to skip a beat for others. In this way, the combined scanning method may be implemented for a range of heart rates.

It may be further desirable to limit the patient's radiation dose to a total predefined level. Thus, if the system is scanning every beat, the mA may be different than if the system is scanning every other beat. The mA could vary on a beat to beat basis by having the system set up for multiple mA profiles, and when an exposure is initiated the system would select the profile associated with the current actual delay. Thus, a CTP exposure after a 2-beat delay could have a different mA profile than a CTP exposure after a 1-beat delay. Alternatively, the system could set up for a 1-beat delay, but as soon as the opportunity to initiate an exposure for that beat is past, the system would update the scan parameters for a 2-beat delay scan. Allowances for other numbers of beats would be a natural extension.

As such, the combined CTP and CTA scanning method described includes multiple phases of scanning, where there is a different mA or delay between scans in each of the phases. The transitions between these phases can be determined in multiple ways. As an example, the time from the start of a scan as determined by a priori information such as a timing bolus may be used to determine the transitions. As a second example, real time computation based on scan data may be used to determine contrast arrival/departure, and be further used to determine transition times. As a third example, transitions may be based on a number of scans in a phase. For example, when a maximum number of scans in a phase is reached, a delay may be triggered until the start of the next phase. As a fourth example, manual intervention by the operator based on real time display of the images may override any of the above automatic transitions that are prescribed.

While interleaving CTA scans within CTP scans, it may be noted that there are one or more settings that may be different or maintained between the scans. For example, the current setting for CTA is typically higher than the current setting for the CTP.

With regard to phase timing, the CTP phase is based on achieving optimal imaging conditions for the ventricular myocardium, and the CTA phase is based on achieving an optimal imaging condition for the coronary arteries. For a combined scan protocol, the CTA exposure may include the CTP phase(s), with the mA during the CTP phase at least equal to the mA of the CTP exposures.

With regard to energy settings, for example, the CTP scans may be at 80 kVp, and the nominal CTA scan may be a dual-energy 80 kVp/140 kVp scan with rapid kVp switching for every view. In this case, the CTA scan may be modified to incorporate scanning during one phase at 80 kVp, such as around 45% of the R-to-R interval, then at around 60% of the R-to-R interval starting to rapidly switch the kVp to acquire a dual-energy scan at around 75%. Different combinations may be used. If the same phase is desired for both the nominal CTP and CTA scans, then single-energy reconstructions from dual-energy acquisitions may be made.

With regard to focal spot, some CT systems use a focal spot size that is a function of the applied mA. Smaller mA levels may be done with a smaller focal spot, and higher mA scans with a larger focal spot. However, it can take several seconds for the system to prepare for a different focal spot size, and such a delay may not be desirable or acceptable to maintain high temporal scanning rates for a CTP exam. In this case, the focal spot size required for the high-mA CTA scan may also be used for the CTP scans, even though this size may be larger than would normally be used for these lower-mA scans. The result is that the CTP scans will have lower spatial resolution, but this is typically a fairly small difference, and the CTP scans do not require the high spatial resolution of the CTA scan, so a slight loss of resolution may not be a clinical impairment.

The CTP scans are focused on the ventricular myocardium. The CTA scans require the entire coronary artery tree, from a little superior to the coronary ostia in the aortic root, to the most inferior side of the heart. Thus, it may be possible to scan a smaller range with the CTP scans, then increase the collimation for the CTA scan. To maintain consistency in any geometric-related artifacts within the imaged volumes, it may be desirable to use an asymmetric collimation for the CTP scans, then open the superior blade of the collimator, leaving the table in a fixed location, for the CTA scan. The CTA and CTP scans may have different scan range requirements, for example 140 mm and 110 mm. By using an asymmetric collimation, the geometry for the scan acquisition of the bottom of the heart can be maintained for all scan acquisitions.

With regard to view count, the CTP scan may have a lower spatial resolution, the CTP scan may have a lower view count, or may use sparse views with the mA turned off or reduced between views. The CTA scan may have a higher view count. The CTP image that is derived from the CTA exposure may only use a subset of the CTA data, or may use a filtered version of the CTA data, or may use the full-fidelity of the CTA data that is acquired during the preferred CTP phase.

In alternate embodiments, in addition to dual energy, the CTA acquisition frame may be at a different kVp (100, for example) than the CTP frames (typically 80 kVp). When kVp is held constant, it may be possible to ramp up and ramp down a frame around the projected optimal CTA frame. As such, the ramping up and ramping down may be considered as a hedge for added robustness, or for additional clinical capability such as coronary flow information. For example, CTP frames may be at, say, 50 mA, however optimal CTA frame may be at 500 mA. In this example, the frame on either side may be at, say 300 mA. Herein, three frames may be utilized for flow analysis, or other post-processing analysis (DSA, for example) and the CTA frame might or might not be included in the CTP analysis. The CTA and CTP phases may be other than as described above. For example, the CTA could be 40-80% with the CTP being either 45% or 75%. Other phase values or ranges could be used. One of the acquired frames, either the CTA or a CTP frame, may acquire a full heart cycle such that LV/RF function and/or valve assessment can be supported from one acquisition sequence as well. Dedicated post processing software may directly process the CTA/CTP hybrid dataset. In this case, the CTA frame might be the best “reference” frame from which frame-to-frame registration is performed prior to the dynamic perfusion analysis.

Multiple ROIs may be used two determine when to transition from one portion of the exposure sequence to another, using either combinatorial or sequential logic. For example, combinational logic (ROI values at 2 distinct locations at the same time point in the scan sequence) may be used as opposed to just a simple sequence from using the ROI values at one location and then the ROI values at a 2nd location.

The bowtie selection may also change for the CTA scan, with the CTP analysis incorporating flexibility for this change.

Thus an example system may include an x-ray source that emits a beam of x-rays toward an object to be imaged; a detector that receives the x-rays attenuated by the object; a data acquisition system (DAS) operably connected to the detector, and a computer operably connected to the DAS and configured with instructions in non-transitory memory that when executed cause the computer to while performing a first scan of a heart of the object, process heart rate data to measure a current interval between successive heart beats, predict a future interval based on the current interval, and determine a trigger time for each of the first scan and a second scan.

Additionally or alternatively, the trigger time may include a first trigger point for the first scan, and further include a second trigger point for the second scan. Additionally or alternatively, the computer may be further configured with instructions in the non-transitory memory that when executed cause the computer to determine each of the first trigger point and the second trigger point based on one or more of a number of scans, a contrast level, the current interval, and the future interval. Additionally or alternatively, the first scan may include a series of perfusion scans performed at a first current setting of the x-ray source, and the second scan may include a single or series of angiography scans performed at a second current setting of the x-ray source, the first current setting being lower than the second current setting. Additionally or alternatively, the computer may be configured with instructions in the non-transitory memory that when executed cause the computer to perform each of the first scan and the second scan using asymmetric collimation of the x-ray source.

FIG. 6 shows a set of graphs 600 illustrating example operating conditions during a scan performed in accordance an embodiment of the invention. The set of graphs includes a plot 505 of measure contrast level over time, a plot 515 of source current in mA, a plot 525 of measured ECG output of a patient.

At time T0, the user may perform a baseline CTP scan as shown by plot 515 and further inject the contrast and start the sequence of scans. Herein, the transition from one inter-scan delay to another inter-scan delay, or from a CTP to a CTA scan, is determined by the system based on a metric derived from the immediately prior scan, or from a sequence of prior scans. The metric may be based on the average CT number within a ROI that is user-placed or algorithmically placed on a baseline image.

At time T1, say a certain time after the contrast injection, the system may perform a sequence of CTP scan (515) every heartbeat while continuously monitoring the ECG data (plot 525) at a first current setting. The perfusion acquisition comprises a series of scans occurring at every heart beat while the measured contrast level increases. Between T1 and T2, the heart rate is regular, however, between T2 and T4, the heart beat is not regular. The system may be able to predict the heart rate changes based on scan analysis performed on a prior set of scans. Based on the predicted heart rate between T2, and T4, the system may be able to re-adjust the timing parameters of the CTP scan in order to be able to scan every beat. However, if the system determines that the heart rate may be too fast to follow, the system may re-adjust the scan interval to a more optimal interval. In some examples, the user may be able to adaptively adjust the interval of scanning. By periodically performing scans while the contrast perfuses through the patient (as illustrated by the measured contrast level in 505), the acquired perfusion data may be used to generate a perfusion map illustrating the perfusion of contrast through the patient.

After a threshold time (time T5, say) is elapsed, the CTP scan may be interrupted by an angiography (CTA) scan. In some examples, when the measured contrast level as shown by plot 505 reaches the threshold Th3, the CTP acquisition may be interrupted and the angiography scan may be performed at T5. In some more examples, when a threshold number of perfusion scans (say 10, for example) is completed, the system may interrupt the perfusion scans and the angiography scan may be performed. In still more examples, a user may interrupt the perfusion scans, and request an angiography scan to be performed at time T5.

Thus, at time T5, the angiography scan may be performed with a second current setting, the second setting being higher than the first setting for the perfusion scans, for example. Upon completion of the angiography scan, the system may continue to perform perfusion scans at every other heartbeat, for example, as shown by plots 515 and 525.

At time T6, the contrast level drops below threshold Th2 (plot 505). The system may begin to perform the perfusion scans at every fourth heartbeat, for example, as shown by plots 515 and 525. As described earlier, in some examples, when a threshold number of perfusion scans performed at every other heart beat is completed, the system may transition to the CTP scan every fourth beat. In still more examples, a user may interrupt and change the inter-scan delay of the perfusion scans.

Responsive to the measured contrast level reaching a minimum threshold, and/or after the completion of a threshold number of CTP scans every fourth beat, the perfusion acquisition ends at time T7. In some examples, the user may intervene and stop the acquisition.

A technical effect of the disclosure is the interleaving of multiple scan protocols within a single dynamic scan session, based on one or more of a contrast level and the heart rate. Another technical effect of the disclosure is the shorter exam times (thus reduced resource utilization and cost per examination). Yet another technical effect of the disclosure is the performance of perfusion and angiography exams with the use of lower radiation dosage. Another technical effect of the disclosure is the reduced cross-contamination of contrast between scans, and hence better quality exams. Another technical effect of the disclosure is the commercial advantage of reduced costs and more saving, and improved patient care.

Various systems and methods for dynamically adapting an imaging scan are provided. In one embodiment, a method comprises, during a scan session, performing a first scan of a heart of a subject using a first scan protocol, performing a second scan of the heart using a second scan protocol, and performing a third scan of the heart using the first scan protocol, and while performing the first scan and the third scan, adjusting a scan rate of the first scan protocol based on a heart rate of the subject.

In a first example of the method, the method includes transitioning from the first scan to the second scan when one or more of a threshold number of scans using the first scan protocol are completed, a threshold time has elapsed, and a threshold contrast level is reached, wherein the contrast level is measured using acquired projection data. In a second example of the method optionally including the first example, the first scan includes multiple perfusion scans performed at different scan rates, and wherein transitioning between the multiple perfusion scans is based on one or more of a scan analysis, the contrast level, and a user input. In a third example of the method optionally including one or more of the first and the second examples, the method further comprises wherein the scan analysis comprises an analysis of a sequence of prior perfusion scans. In a fourth example of the method optionally including one or more of the first through third examples, the first scan protocol includes a first current setting of a source of the scanner. In a fifth example of the method optionally including one or more of the first through fourth examples, the second scan comprises an angiography scan, and the second scan protocol includes a second current setting of the source of the scanner, the first current setting lower than the second current setting.

In another representation, a method comprises: while performing a first scan of a heart of a subject at a first interval, processing acquired projection data to measure a contrast level; responsive to the contrast level increasing above a first threshold, performing the first scan at a second interval for a threshold time; intermittently performing a second scan upon completion of the threshold time and resuming the first scan at the second interval; and responsive to the contrast level decreasing below a second threshold, performing the first scan at a third interval, each of the first interval, the second interval and the third interval adjusted based on a heart rate of the subject.

In a first example of the method, the method includes performing the second scan after completion of a threshold number of the first scan at the second interval, and based on a user input. In a second example of the method optionally including the first example, and further includes performing the second scan responsive to the contrast level increasing above a third threshold, the third threshold being higher than the first threshold and the second threshold. In a third example of the method optionally including one or more of the first and the second examples, the method further comprises determining an interval of the second scan based on the heart rate and further adjusting the interval based on one or more of a scan analysis and the user input, the scan analysis including analysis of sequence of prior scans. In a fourth example of the method optionally including one or more of the first through third examples, the method comprises adjusting the first interval, second interval, and third interval based on one or more of the scan analysis, the user input, and an inter-scan delay determined based on the heart rate. In a fifth example of the method optionally including one or more of the first through fourth examples, and further wherein the first scan includes a series of perfusion scans performed at a first current setting of a source of the scanner, and the second scan includes a series of angiography scans performed at a second current setting of the source of the scanner, the first current being lower than the second current.

In another embodiment, a non-transitory computer-readable storage medium includes executable instructions stored thereon that when executed by a computer cause the computer to: start a sequence of a first set of perfusion scans of a heart of a patient with a first inter-scan interval; responsive to completion of a first threshold number of the first set of perfusion scans, perform a second set of perfusion scans with a second inter-scan interval, wherein during the second set of perfusion scans, the instructions further cause the computer to: monitor contrast level of an injected contrast agent based on projection data acquired during the second set of perfusion scans responsive to the contrast level above a threshold, interleave a set of angiography scans for a threshold duration between the second set of perfusion scans; responsive to completion of the threshold duration, resume the second set of perfusion scans; responsive to completion of the second threshold number of the second set of perfusion scans, perform a third set of perfusion scans with a third inter-scan interval for a threshold time; end scan session upon completion of the threshold time; and reconstruct at least one diagnostic image based on one or more of sets of perfusion scans and sets of angiography scans.

In a first example of the non-transitory computer-readable storage medium, the instructions further cause the computer to: calculate each of the first inter-scan interval, the second inter-scan interval, and the third inter-scan interval based on an inter-beat interval of the heart of the patient. In a second example of the non-transitory computer-readable storage medium optionally including the first example, wherein the first inter-scan interval is lower than each of the second inter-scan interval, and the third inter-scan interval, and further wherein the second scan interval is lower than the third scan interval. In a third example of the non-transitory computer-readable storage medium optionally including one or more of the first and second examples, wherein the instructions further cause the computer to: interleave the set of angiography scans upon completion of a third threshold number of the second set of perfusion scans, the third threshold number being lower than the second threshold number. In a fourth example of the non-transitory computer-readable storage medium optionally including one or more of the first through third examples, wherein the instructions further cause the computer to: determine the third threshold number based on one or more of an immediately prior scan and a sequence of prior scans of the second set of perfusion scans. In a fifth example of the non-transitory computer-readable storage medium optionally including one or more of the first through fourth examples wherein the instructions further cause the computer to: interleave the set of angiography scans at a time point determined based on or more of scan data analysis, and a user input. In a sixth example of the non-transitory computer-readable storage medium optionally including one or more of the first through fifth examples, wherein the instructions further cause the computer to: determine each of the first threshold number and the second threshold number based on one or more of the scan data analysis and the user input. In a seventh example of the non-transitory computer-readable storage medium optionally including one or more of the first through sixth examples, further wherein the instructions further cause the computer to: determine the threshold time based on one or more of the scan data analysis and the user input. In an eighth example of the non-transitory computer-readable storage medium optionally including one or more of the first through seventh examples, further wherein the instructions further cause the computer to: perform the set of angiography scans at a higher current setting of a source than each of the first set, the second set and the third set of perfusion scan.

In yet another embodiment, a system comprises: an x-ray source that emits a beam of x-rays toward an object to be imaged; a detector that receives the x-rays attenuated by the object; a data acquisition system (DAS) operably connected to the detector; and a computer operably connected to the DAS and configured with instructions in non-transitory memory that when executed cause the computer to: while performing a first scan of a heart of the object, process heart rate data to measure a current interval between successive heart beats; predict a future interval based on the current interval; and determine a trigger time for each of the first scan and a second scan.

In a first example of the system, the trigger time may include a first trigger point for the first scan, and may further include a second trigger point for the second scan. In a second example of the system optionally including the first example, wherein the computer is further configured with instructions in the non-transitory memory that when executed cause the computer to determine each of the first trigger point and the second trigger point based on based on one or more of a number of scans, a contrast level, the current interval and the future interval. In a third example of the system optionally including one or more of the first and second examples, the system further includes wherein the first scan includes a series of perfusion scans performed at a first current setting of the x-ray source, and the second scan includes a series of angiography scans performed at a second current setting of the x-ray source, the first current setting being lower than the second current setting. In a fourth example of the system optionally including one or more of the first through third examples, and further wherein the computer is further configured with instructions in the non-transitory memory that when executed cause the computer to perform each of the first scan and the second scan using asymmetric collimation of the x-ray source.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
during a scan session, performing a first scan of a heart of a subject using a first scan protocol, performing a second scan of the heart using a second scan protocol, and performing a third scan of the heart using the first scan protocol; and
while performing the first scan and the third scan, adjusting a scan rate of the first scan protocol based on a heart rate of the subject.

2. The method of claim 1, further comprising transitioning from the first scan to the second scan when one or more of a threshold number of scans using the first scan protocol are completed, a threshold time has elapsed, and a threshold contrast level is reached, wherein contrast level is measured using acquired projection data.

3. The method of claim 2, wherein the first scan includes multiple perfusion scans performed at different scan rates, and wherein transitioning between the multiple perfusion scans is based on one or more of a scan analysis, the contrast level, and a user input.

4. The method of claim 3, wherein the scan analysis comprises an analysis of a sequence of prior perfusion scans.

5. The method of claim 3, wherein the first scan protocol includes a first current setting of a source.

6. The method of claim 5, wherein the second scan comprises an angiography scan, and wherein the second scan protocol includes a second current setting of the source, the first current setting lower than the second current setting.

7. A non-transitory computer-readable storage medium including executable instructions stored thereon that when executed by a computer cause the computer to:
start a sequence of a first set of perfusion scans of a heart of a patient with a first inter-scan interval;
responsive to completion of a first threshold number of the first set of perfusion scans, perform a second set of perfusion scans with a second inter-scan interval, wherein during the second set of perfusion scans, the instructions further cause the computer to:
monitor contrast level based on projection data acquired during the second set of perfusion scans;
responsive to the contrast level above a threshold, interleave a set of angiography scans for a threshold duration between the second set of perfusion scans;
responsive to completion of the threshold duration, resume the second set of perfusion scans;
responsive to completion of a second threshold number of the second set of perfusion scans, perform a third set of perfusion scans with a third inter-scan interval for a threshold time;
end scan session upon completion of the threshold time; and
reconstruct at least one diagnostic image based on one or more of sets of perfusion scans and sets of angiography scans.

8. The non-transitory computer-readable storage medium of claim 7, wherein the instructions further cause the computer to: calculate each of the first inter-scan interval, the second inter-scan interval, and the third inter-scan interval based on an inter-beat interval of the heart of the patient.

9. The non-transitory computer-readable storage medium of claim 8, wherein the first inter-scan interval is lower than each of the second inter-scan interval, and the third inter-scan interval, and further wherein the second inter-scan interval is lower than the third inter-scan interval.

10. The non-transitory computer-readable storage medium of claim 7, wherein the instructions further cause the computer to: interleave the set of angiography scans upon completion of a third threshold number of the second set of perfusion scans, the third threshold number being lower than the second threshold number.

11. The non-transitory computer-readable storage medium of claim 10, wherein the instructions further cause the computer to: determine the third threshold number based on one or more of an immediately prior scan and a sequence of prior scans of the second set of perfusion scans.

12. The non-transitory computer-readable storage medium of claim 7, wherein the instructions further cause the computer to: interleave the set of angiography scans at a time point determined based on or more of scan data analysis, and a user input.

13. The non-transitory computer-readable storage medium of claim 12, wherein the instructions further cause the computer to: determine each of the first threshold number and the second threshold number based on one or more of the scan data analysis and the user input.

14. The non-transitory computer-readable storage medium of claim 13, wherein the instructions further cause the computer to: determine the threshold time based on one or more of the scan data analysis and the user input.

15. The non-transitory computer-readable storage medium of claim 7, wherein the instructions further cause the computer to: perform the set of angiography scans at a higher current setting of a source than each of the first set, the second set and the third set of perfusion scans.

16. A system, comprising:
an x-ray source that emits a beam of x-rays toward an object to be imaged;
a detector that receives the x-rays attenuated by the object;
a data acquisition system (DAS) operably connected to the detector; and
a computer operably connected to the DAS and configured with instructions in non-transitory memory that when executed cause the computer to:
while performing a first scan of a heart of the object, process heart rate data to measure a current interval between successive heart beats;
predict a future interval based on the current interval; and
determine a trigger time for each of the first scan and a second scan.

17. The system of claim 16, wherein the trigger time includes a first trigger point for the first scan, and further includes a second trigger point for the second scan.

18. The system of claim 17, wherein the computer is further configured with instructions in the non-transitory memory that when executed cause the computer to determine each of the first trigger point and the second trigger point based on one or more of a number of scans, a contrast level, the current interval and the future interval.

19. The system of claim 18, wherein the first scan includes a series of perfusion scans performed at a first current setting of the x-ray source, and the second scan includes a series of angiography scans performed at a second current setting of the x-ray source, the first current setting being lower than the second current setting.

20. The system of claim 19, wherein the computer is further configured with instructions in the non-transitory memory that when executed cause the computer to perform each of the first scan and the second scan using asymmetric collimation of the x-ray source.
